(19) United States
(12) Patent Application Publication
(10) Pub. No.: US 2009/0181973 A1
(43) Pub. Date: Jul. 16, 2009

(54) Medicinal composition comprising 3-alkoxy-6-allylthiopyridazines for prevention or treatment of cancer of the pancreas

(76) Inventors: Soon-Kyoung Kwon, Seoul (KR); Yong-Soo Lee, Dobong-gu Seoul (KR); Hae-Sun Park, Seoul (KR)

Correspondence Address: Kenneth I. Kohn;Kohn & Associates, 30500 Northwestern Highway, Suite 410, Farmington Hills, MI, 48334, US

(21) Appl. No.: 11/596,428

(22) PCT Filed: Nov. 16, 2005
(86) PCT No.: PCT/KR05/03879

§ 371 Date: Nov. 10, 2006

(51) Int. Cl. A61K 31/50; C07D 237/18; A61P 35/00

(52) U.S. Cl. 514/247; 544/240

(57) ABSTRACT

The present invention relates to a medicinal composition for preventing or treating pancreatic cancer, comprising 3-alkoxy-6-allylthiopyridazine or a pharmaceutically acceptable salt thereof as an effective ingredient. An MTT assay, to assess the viability of cells, and flow cytometric analysis, to measure apoptotic cell death, were performed, and the results, which revealed that the compound and pharmaceutically acceptable salts thereof have preventive and therapeutic activity against pancreatic cancer, are presented herein.

### TECHNICAL FIELD

The present invention relates to a medicinal composition for preventing or treating pancreatic cancer comprising 3-alkoxy-6-allylthiopyridazine, represented by Chemical Formula I, below, or a pharmaceutically acceptable salt thereof as an effective ingredient, and a method of preparing the compound. The present invention is also concerned with a method of treating pancreatic cancer using the compound of Chemical Formula I, and the use of the compound of Chemical Formula I in the preparation of a medicament for preventing or treating pancreatic cancer.

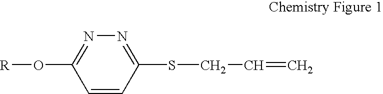

Wherein, R represents —CH₃, —C₂H₅, —C₃H₇ or —CH(CH₃)₂.

### BACKGROUND ART

Edible organic sulfur compounds including diallylsulfide, which is a component of garlic oil, have been known to inhibit the proliferation of tumor cells and suppress chemically induced carcinogenesis in several organs in experimental animals. Some synthetic sulfur-containing compounds, including oltipraz and sulindac, have been revealed to exhibit chemical prophylactic activity in laboratory studies on carcinogenesis. Besides, although pyridazines were discovered a long time ago (1886), they did not attract particular interest compared to two chemically similar compounds, pyrimidines and pyrazines. Recently, many efforts have been made to identify the biological and pharmacological activities of pyridazine derivatives. Pyridazines and their derivatives have been reported to exhibit various activities including the production of reactive oxygen species, induction of hepatic microsomal enzymes, and inhibition of tumors.

The identification and development of pharmaceutical agents capable of selectively regulating an apoptotic pathway may be effective strategies for the prevention or treatment of cancer. Some evidence suggests that the activation of caspases induces the apoptosis process in several types of cells. Caspase-3 has been shown to play a fundamental role in apoptosis induced by various causes. According to a recent report, a possible mechanism of specified pro-caspase-3 activation involves the release of respiratory cytochrome c from the mitochondria into the cytoplasm. In the cytoplasm, cytochrome c forms a complex which results in the activation of pro-caspase-9, which in turn, cleaves pro-caspase-3 to convert it to an active form. The anti-apoptotic Bcl-2 oncoprotein acts on the mitochondria to block the release of cytochrome c, thereby preventing caspase activation (Sundaram S. G., Milner J. A., Diallyl disulfide inhibits the proliferation of human tumor cells in culture, Biochem Biophys Acta, 1996, 1315, pp 15-20; Siegers C. P., Steffen B., Robke A., Pentz R., The effects of garlic preparations against human tumor cell proliferation, Phytomedicine 1999, 6, pp 7-11; Fukushima S., Takada N., Hori T., Wanibuchi H., Cancer prevention by organosulfur compounds from garlic and onion, J. Cell Biochem Suppl., 1997, 27, pp 100-105; Reddy B. S., Rao C. V., Rivenson A., Kelloff G., Chemoprevention of colon carcinogenesis by organosulfur compounds, Cancer Res., 1993, 53, pp 3493-3498; Rahman M. A., Dhar D. K., Masunaga R. M., Yamanoi A., Kohno H., Nagasue N., Sulindac and exisulind exhibit a significant antiproliferative effect and induce apoptosis in human hepatocellular carcinoma cell lines, Cancer Res., 2000, 60, pp 2085-2089; Porter A. G., Janike R. U., Emerging roles of caspase-3 in apoptosis, Cell Death Differ, 1999, 6, pp 99-104; Kluck R. M., Bossy-Wetzel E., Green D. R., Newmeyer D. D., The release of cytochrome c from mitochondria: a primary site for bcl-2 regulation of apoptosis, Science, 1997, 275, pp 1132-1136; Nicholson D. W., Ali A., Thornberry N. A., et al., Identification and Inhibition of the ICE/CED-3 protease necessary for mammalian apoptosis, Nature, 1995, 376, pp 37-43; Lee E., Kong G., Lee S. J., Kim N. D., Surh Y. J., 2-(Allylthio)pyrazine suppresses the growth and proliferation of human promyelocytic leukemia (HL-60) cells via induction of apoptosis, Anticancer Res., 1999, 19, pp 4073-4080; Oltvai Z. N., Milliman C. L., Korsmeyer S. J., Bcl-2 heterodimerizes in vivo with a conserved homolog, Bax, that accelerates programmed cell death, Cell, 1993, 74, pp 609-619).

### DISCLOSURE OF INVENTION

### Technical Problem

Accordingly, the present invention aims to provide a medicinal composition for use as a preventive or therapeutic agent for pancreatic cancer and a method of preparing such a composition, a method of treating pancreatic cancer using the compound of Chemical Formula I, and the use of the compound of Chemical Formula I in the preparation of a medicament for preventing or treating pancreatic cancer.

### Technical Solution

Based on the background, the present inventors made many efforts to find compounds which are capable of preventing or treating particularly liver cancer by effectively acting on the known mechanism for the prevention or treatment of cancer. The present inventors found that among the compounds of Chemical Formula II, below, which were developed as hepatic protectors by the present inventors (see, Korean Pat. Application No. 1998-0001153 filed on Jan. 16, 1998), the compound of Chemical Formula I, below, a 3-alkoxy-6-allylthiopyridazine derivative, is capable of effectively preventing or treating liver cancer (see, Korean Pat. Application No. 2002-0039955 filed on Jul. 10, 2002). The present inventors continued studies to determine whether these compounds are useful as therapeutic agents for types of cancer other than liver cancer. As a result, the compound of Chemical Formula I was also found to be capable of effectively preventing or treating pancreatic cancer through its action of inhibiting the growth of pancreatic carcinoma cells, thereby leading to the present invention. Unlike other types of cancer, effective therapeutic agents for pancreatic cancer have not been developed so far. For this reason, pancreatic cancer has a very high mortality rate relative to its incidence. Thus, there is an urgent need for the development of effective therapeutic agents for pancreatic cancer. Therefore, the ability of 3-alkoxy-6-allylthiopyridazine to effectively inhibit the growth of pancreatic carcinoma cells, indicating that this compound can be used as an effective preventive or therapeutic agent for pancreatic cancer, is an unpredicted and surprising finding.

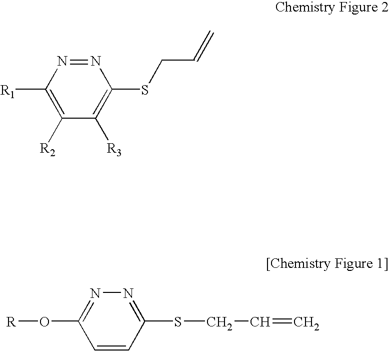

Wherein, R represents —CH₃, —C₂H₅, —C₃H₇ or —CH(CH₃)₂,

R₁ represents a halogen atom, or a lower alkoxy, dialkylaminoalkoxy, hydroxyalkoxy, or phenoxy, benzyloxy or phenyl substituted or unsubstituted with lower alkyl, and

R₂ and R₃ are each independently hydrogen or a lower alkyl, or R₂ and R₃ may form a saturated or unsaturated six-membered ring along with carbon atoms to which they are attached,

in which R₂ and R₃ are not hydrogen when R₁ is chloro.

### BRIEF DESCRIPTION OF THE DRAWINGS

FIG. 1 shows the results of the measurement of viable cell number using an MTT assay when pancreatic carcinoma cells were treated with 3-alkoxy-6-allylthiopyridazine (also referred to simply as K-6) according to the present invention. An open circle indicates BxPC3 pancreatic carcinoma cells, and an open square indicates PANC1 pancreatic carcinoma cells. Data are expressed as mean±SD of four independent experiments under the same conditions.

FIG. 2 shows the results of the measurement of apoptotic cell death in BxPC3 pancreatic carcinoma cells treated with the K-6 compound according to the present invention. BxPC3 cells were stained with PI, and apoptotic cell death was measured using flow cytometric analysis. A cell population undergoing apoptosis, designated A₀, consists of cells which are distributed below the G₀/G₁ peak, wherein G₀ and G₁ indicate a population at the resting stage of cell division. Data shown herein are the results of representative flow cytometric analysis.

FIG. 3 shows the results of the measurement of apoptotic cell death in pancreatic carcinoma cells treated with the K-6 compound according to the present invention. Pancreatic carcinoma cells were stained with PI, and apoptotic cell death was measured using flow cytometric analysis. A black bar indicates BxPC3 pancreatic carcinoma cells, and a white bar indicates PANC1 pancreatic carcinoma cells. Data are expressed as mean±SD of four independent experiments under the same conditions.

### BEST MODE FOR CARRYING OUT THE INVENTION

In one aspect, the present invention relates to a medicinal composition for preventing or treating pancreatic cancer, comprising 3-alkoxy-6-allylthiopyridazine of Chemical Formula I, below, or a pharmaceutically acceptable salt thereof as an effective ingredient.

In another aspect, the present invention relates to a method of preventing or treating pancreatic cancer, comprising administering a therapeutically effective amount of 3-alkoxy-6-allylthiopyridazine of Chemical Formula I, below, or a pharmaceutically acceptable salt thereof to a mammal.

In a further aspect, the present invention relates to a method of preparing a pharmaceutical composition for preventing or treating pancreatic cancer, comprising mixing 3-alkoxy-6-allylthiopyridazine of Chemical Formula I, below, or a pharmaceutically acceptable salt thereof as an effective ingredient with a pharmaceutically acceptable carrier.

In yet another aspect, the present invention relates to the use of 3-alkoxy-6-allylthiopyridazine of Chemical Formula I, below, or a pharmaceutically acceptable salt thereof in the preparation of a medicament for preventing or treating pancreatic cancer.

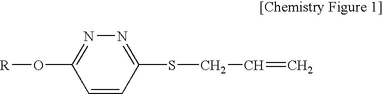

Wherein, R represents —CH₃, —C₂H₅, —C₃H₇ or —CH(CH₃)₂, and is preferably —CH₃.

The present inventors synthesized 3-alkoxy-6-allylthiopyridazine using pyridazine as a parent compound. In detail, 3-alkoxy-6-chloropyridazine was primarily prepared from 3,6-dichloropyridazine, and this halogenated compound was allowed to react with allylmercaptane to provide 3-alkoxy-6-allylthiopyridazine in a very pure form. The finally synthesized compound was characterized using NMR and FTIR. The finally synthesized compound was then tested for anticancer activity against two pancreatic carcinoma cell lines, BxPC3 and PANC1.

Pharmaceutically acceptable salts of the 3-alkoxy-6-allylthiopyridazine compound include pharmaceutically acceptable acid addition salts, such as aspartate, gluconate, glutamate, chlorate, para-toluenesulfonate or citrate, salts of alkali metals, such as sodium, potassium or lithium, and salts of other known acids or bases used in compounds such as oltipraz, diallylsulfide, or allicin. These pharmaceutically acceptable salts are prepared through ordinary conversion processes.

In order to determine whether the 3-alkoxy-6-allylthiopyridazine compound has anticancer activity against two pancreatic carcinoma cell lines, BxPC3 and PANC1, the present inventors conducted an MTT assay to assess the viability of cells and flow cytometric analysis to measure apoptotic cell death. As a result, the present compound was found to have excellent preventive or therapeutic effects on pancreatic cancer through its action of inhibiting the growth of pancreatic carcinoma cells.

For clinical administration, the compound of Chemical Formula I contained in the medicinal composition according to the present invention may be admixed with a pharmaceutically-acceptable inert carrier and formulated into a dosage form in a solid, semi-solid or liquid form suitable for oral or parenteral administration.

A suitable pharmaceutically-acceptable inert carrier for use for such a purpose may be in the form of solid or liquid, and may be one or more selected from among diluents, fragrances, solubilizers, lubricants, suspending agents, binders, and materials serving as bulking agents for tablets. Detailed examples of solid or liquid carriers suitable for use in the present invention include starch, lactose, cellulose derivatives (e.g., Avicel), and sucrose.

For use for preventive or therapeutic purposes against pancreatic cancer, the medicinal composition of the present invention is preferably administered initially in a dosage of about 1 to 500 mg of active compound per kg of body weight per day. However, it is understood by those skilled in the art that the dosage may vary depending on the patients need, the severity of the condition to be treated, and the type of compound to be used, and that a desirable dosage may be determined for a specific case. Therapy usually begins with a dosage less than an optimal amount. The initial dosage is then increased gradually according to the situation until the optimal response is achieved. For convenience, the daily dosage may be divided into doses taken several times a day.

From preliminary in vivo safety data for acute oral toxicity, no abnormal finding was detected in rodents received a dosage of 1.3 mmol/kg/day of 3-alkoxy-6-allylthiopyridazine.

### MODE FOR THE INVENTION

A better understanding of the present invention may be obtained through the following examples which are set forth to illustrate, but are not to be construed as the limit of the present invention.

### Test Example 1

### Culture of Pancreatic Carcinoma Cells

Human pancreatic carcinoma cell lines, BxPC3 and PANC1, which were purchased from the American Type Culture Collection (ATCC), were cultured in Dulbecco's Modified Eagle's Medium (DMEM; Gibco BRL, Grand Island, N.Y., USA) supplemented with 10% fetal bovine serum (FBS; Gibco BRL, Grand Island, N.Y., USA) and 1% penicillin-streptomycin (Gibco BRL, Grand Island, N.Y., USA) to achieve a density of 5 10⁵ cells/ml. Cells were cultured in monolayer in 75 mm² flasks containing 15 ml of the medium in a humidified incubator with 5% CO₂ at 37?. The medium was changed twice a week. Once cells reached confluency, they were subcultured by trypsinization with 0.05% trypsin-EDTA.

### Test Example 2

### Measurement of Viable Cell Number Using MTT Assay

Viable cells convert 3-[4,5-dimethylthiazol-2-yl]-2,5-diphenyl tetrazolium bromide (MTT) into MTT formazan using mitochondrial dehydrogenase. MTT transformation is directly proportional to the number of viable cells. Pancreatic carcinoma cells were dosed only with 3-alkoxy-6-allylthiopyridazine (hereinafter, referred to simply as K-6) in 1 ml medium in 24-well multiwell plates, and cultured for 48 hrs. Then, 100□ of an MTT solution (2.5 g/ml H₂O) were added to each well, followed by incubation at 37° C. for 4 hrs. To measure the concentration of MTT formazan crystals formed, each cell suspension thus treated was transferred into an Eppendorf tube and centrifuged at 1,500 rpm for 4 min. After the supernatant was carefully discarded, 100□ of dimethyl sulfoxide (DMSO) was added to the tube in order to dissolve the MTT formazan crystals. Absorbance was measured at 540 nm using an ELISA leader (Molecular Devices, California, USA). As a control, cells were treated according to the same procedure except that the drug (K-6) was not added, and the absorbance of MTT formazan was measured.

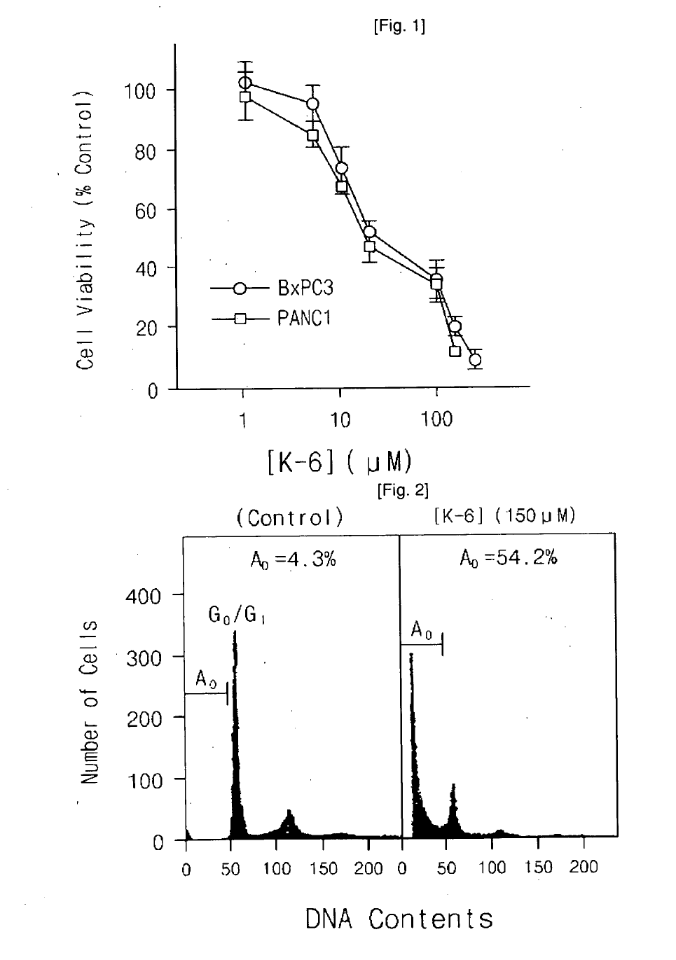

Cell viability (%) was calculated according to the following equation: Cell viability (%)=(Ab. of drug-treated cells/Ab. of control) 100%, and was expressed as a graph (FIG. 1).

### Test Example 3

### Quantization of Apoptotic Cell Death Using Flow Cytometric Analysis

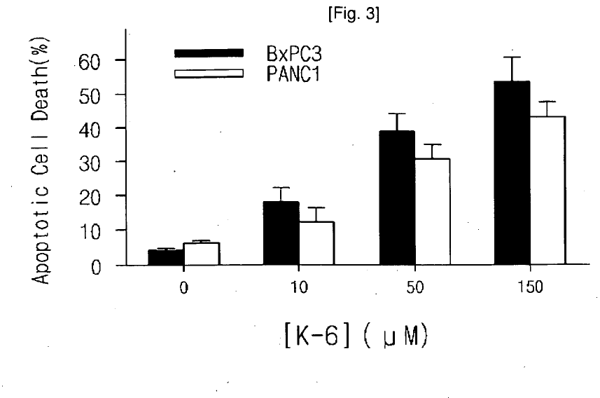

Since cells undergoing apoptosis lose fragmented DNA, the number of cells possessing sub-G₁ DNA was measured using flow cytometric analysis in order to determine the extent of apoptotic cell death. Pancreatic carcinoma cells were dosed with the drug (K-6) and grown for two days. Cells were then washed with PBS, and detached from a culture container by trypsinization. After cells were centrifuged at 1,200 rpm, the cell pellet was washed with a 1:1 mixture of PBS and McIlvaine's buffer (0.2 M Na₂HPO₄, 0.1 M citric acid, pH 7.5). Two volumes of ethanol pre-chilled to 4° C. was added to and gently mixed with the washed cells in order to fix the cells. The fixed cells were suspended in a 4 mM sodium citrate solution containing 0.1% Triton X-100, 32□/ml RNase A and 50□/ml propidium iodide (PI), and were incubated at 4° C. for more than 16 hrs. The DNA content of the cells was analyzed using a flow cytometer (BIO-RAD, California, USA). As a control, pancreatic carcinoma cells were treated according to the same procedure except that the drug (K-6) was not added, and the DNA content of the control was measured (FIGS. 2 and 3).

As apparent from the results of Test Examples 2 and 3, when human pancreatic carcinoma cell lines, BxPC3 and PANC1, were treated with the 3-alkoxy-6-allylthiopyridazine compound for 48 hrs, the number of live cells decreased dependent on the dose of the compound (IC₅₀=about 20 μM). Also, flow cytometric analysis revealed that the 3-alkoxy-6-allylthiopyridazine compound induces apoptotic cell death in pancreatic carcinoma cells.

### INDUSTRIAL APPLICABILITY

Although extensive effort has been made to develop substances for therapeutic use against pancreatic cancer, therapeutic agents having therapeutic effects against pancreatic cancer have not been developed. Therefore, the 3-alkoxy-6-allylthiopyridazine compound according to the present invention, which has an excellent effect of inhibiting the growth of human pancreatic carcinoma cells, is a potential preventive or therapeutic agent against pancreatic cancer.

1. A medicinal composition for preventing or treating pancreatic cancer, comprising 3-alkoxy-6-allylthiopyridazine of Chemical Formula I, or a pharmaceutically acceptable salt thereof as an effective ingredient, along with a pharmaceutically acceptable carrier: 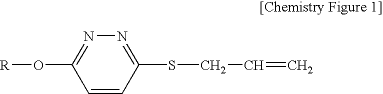
wherein, R represents —CH₃, —C₂H₅, —C₃H₇ or —CH(CH₃)₂.

2. The composition according to claim 1, wherein the R of the compound of Chemical Formula I is —CH₃.

3. A method of preventing or treating pancreatic cancer, comprising administering a therapeutically effective amount of 3-alkoxy-6-allylthiopyridazine of Chemical Formula I, below, or a pharmaceutically acceptable salt thereof to a mammal: 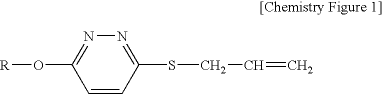
wherein, R represents —CH₃, —C₂H₅, —C₃H₇ or —CH(CH₃)₂.

4. The method according to claim 3, wherein the R of the compound of Chemical Formula I is —CH₃.

5. A method of preparing a pharmaceutical composition for preventing or treating pancreatic cancer, comprising mixing 3-alkoxy-6-allylthiopyridazine of Chemical Formula I or a pharmaceutically acceptable salt thereof as an effective ingredient with a pharmaceutically acceptable carrier: 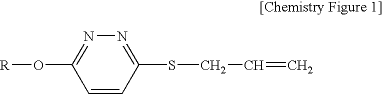
wherein, R represents —CH₃, —C₂H₅, —C₃H₇ or —CH(CH₃)₂.

6. The method according to claim 5, wherein the R of the compound of Chemical Formula I is —CH₃.

7. A use of 3-alkoxy-6-allylthiopyridazine of Chemical Formula I or a pharmaceutically acceptable salt thereof for the preparation of a medicament for preventing or treating pancreatic cancer: 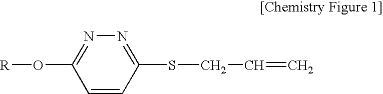
wherein, R represents —CH₃, —C₂H₅, —C₃H₇ or —CH(CH₃)₂.

8. The use according to claim 7, wherein the R of the compound of Chemical Formula I is —CH₃.
